(19) United States
(12) Patent Application Publication
(10) Pub. No.: US 2018/0364211 A1
(43) Pub. Date: Dec. 20, 2018

(54) Soil Analysis Method

(71) Applicant: Injekta Environmental Pty Ltd, Malvern (AU)

(72) Inventors: Edward Dundas Scott, Malvern (AU); Michael Eyres, Malvern (AU)

(21) Appl. No.: 16/061,252

(22) PCT Filed: Dec. 12, 2016
(86) PCT No.: PCT/AU2016/000396

§ 371 Date: Jun. 11, 2018

(30) Foreign Application Priority Data
Dec. 11, 2015 (AU) 2015905128

(51) Int. Cl. G01N 33/24

(52) U.S. Cl. CPC G01N 33/24; G01N 2033/245

(57) ABSTRACT

A method for performing a soil analysis to determine quantities of one or more cations to be included in a soil treatment plan for an agricultural soil is described by either directly measuring or indirectly estimating the Zeta Potential of a soil sample. From these measurements or estimates appropriate quantities of water soluble cations (Ca, Mg, K, Na) can be determined and the Zeta Potential can be adjusted to a desired value. A method of estimating Zeta Potential is obtained from measurements of soluble and exchangeable cation concentrations, which are combined to generate Cation Relationship of Plants and Soil Solution (CROPSS) Value, and then through the use of a mapping relationship between a CROPSS value and Zeta Potential for reference soils, the CROPSS value can be used to determine the appropriate soil treatment to be applied that will adjust the Zeta Potential to a desired range or value.

### PRIORITY DOCUMENT

The present application claims priority from Australian Provisional Patent Application No. 2015905128 titled “SOIL ANALYSIS METHOD” and filed on 11 Dec. 2016, the content of which is hereby incorporated by reference in its entirety.

### TECHNICAL FIELD

The present invention relates to methods for performing soil analysis for agricultural soils. In a particular form the present invention relates to method for performing soil analysis to enable development of a soil treatment plan for agricultural soils.

### BACKGROUND

Management of soils for agricultural production is of great importance. Plant yields depend upon the ability of the plant to uptake nutrients from the soil. These in turn depend on factors such as the physical, chemical and biological properties of the soil, all of which change with time depending upon past usage (eg previous crops and cultivation methods). In particular commercial cultivation of soil rapidly depletes the soil nutrients and thus it is recommended that soil tests be performed regularly (eg yearly, or every 2-3 years). Soil tests provide a snapshot of the nutrients and properties of the soil and so can be used to monitor soil condition and fertility levels, and to be used as a tool to guide the type and amount of fertiliser to apply to optimise plant growth.

Typically a farmer will take one or more soil samples from a site which are sent to a laboratory to perform a soil test which will measure a range of soil parameters to determine the soil fertility and condition, or overall soil health. These soil parameters include quantities of various essential and trace minerals and nutrients such as Nitrogen (N), Phosphorus (P), Potassium (K), Sulphur (S), Calcium (Ca), Magnesium (Mg), Iron (Fe), Zinc (Z), etc. Sodium (Na), Potassium (K), Calcium (Ca). Typically the quantities are determined using acid based assays. Additionally the soil parameters include chemical and physical soil characteristics such as the pH, % water content, % organic carbon content, electrical conductivity (EC),cation exchange capacity (CEC—also known as nutrient holding capacity), and may report on other parameters such as the texture or soil type (eg according to a classification scheme). A soil report is produced and the measured values are compared to with reference or guideline values or ranges to assess soil health so that a suitable treatment plan can be developed and implemented to improve the soil health. The appropriate levels depend upon the soil use or region. For example the Victorian Government Department of Environment and Primary Industries provide recommendations on appropriate levels of typical parameters for Dairy farms (http://www.depi.vic.gov.au/agriculture-and-food/ dairy/pastures-management/fertilising-dairy-pastures/interpreting-soil-and-tissue-tests). Similarly the NSW Department of Primary Industries provides suggestions for the North Coast of NSW (http://www.dpi.nsw.gov.au/agriculture/resources/soils/testing/interpret).

The report can then be used to determine soil management plans that provide recommendations of the type and amount of bulk fertiliser to apply to optimise plant growth. Typically this will involve determining which nutrient (or nutrients) is deficient and by how much. An appropriate quantity of a standard fertiliser composition or a custom fertiliser composition is then selected in order to raise the nutrient(s) to the recommended level(s). Additional soil treatments such as mechanical aeration (coring), or application of soil ameliorants (eg gypsum, clay breakers) to alter the physical/chemical structure of the soil may also be recommended. However simply adding extra quantities of a deficient nutrient to the soil to raise the nutrient to a desired level is often only partially effective.

There is thus a need to provide improved soil analysis methods for determining soil treatment (or management) plans for agricultural soils, or at least to provide a user with a useful alternative to existing methods.

### SUMMARY

According to a first aspect, there is provided a method for performing a soil analysis to determine quantities of one or more cations to be included in a soil treatment plan for an agricultural soil, the method comprising:

measuring a water soluble cation concentration and an exchangeable cation concentration in one or more received soil samples for a first group of cations comprising Calcium and at least one of Magnesium, Potassium and Sodium;

estimating a Cation Relationship of Plants and Soil Solution (CROPSS) value wherein the CROPSS value is defined as a first ratio multiplied by a second ratio, where the first ratio comprises a soluble cation concentration selected from the first group of cations divided by the sum of soluble cation concentrations for each of the first group of cations, and the second ratio comprises an exchangeable cation concentration selected from the first group of cations divided by the sum of exchangeable cation concentrations for each of the first group of cations;

defining either a target CROPSS value or a desired range of CROPSS values, wherein the target CROPSS value or a desired range of CROPSS values are obtained using a predetermined mapping relationship between the CROPSS value and a Zeta Potential, and the target CROPSS value is obtained using a predetermined target Zeta Potential and the mapping relationship, and the lower bound of the desired range of CROPSS values is obtained using a predetermined threshold Zeta Potential and the mapping relationship; and

calculating a quantity of one or more of the water soluble cations from the first group and/or a quantity of one or more of the exchangeable cations from the first group required to result in an adjusted CROPSS value within the predetermined threshold value for inclusion in a treatment plan such that an adjusted CROPSS value calculated using the measured values and added quantities is either equal to the target CROPSS value, or within the desired range of CROPSS values if the compared CROPSS value is outside of the predetermined threshold value.

In a one form, the predetermined mapping relation is obtained by performing a regression of CROPSS values against Zeta Potential measurements for a range of soils.

In a one form, the predetermined Zeta Potential threshold is −30, and according to the mapping relationship the adjusted CROPSS value maps to a Zeta Potential greater than −30.

In one form, the estimated CROPSS value is outside of the predetermined threshold value if the estimated CROPSS value is less than lower threshold CROPSS value. In a further form, the first group comprises Calcium and each of Magnesium, Potassium and Sodium, and the lower threshold CROPSS value is 25.5, and the first ratio comprises the soluble Calcium cation divided by the sum of soluble cation concentrations for each of the first group of cations, and the second ratio comprises the exchangeable Calcium cation concentration divided by the sum of exchangeable cation concentrations for each of the first group of cations.

In one form the step of calculating a quantity of one or more of the water soluble cations from the first group and/or a quantity of one or more of the exchangeable cations from the first group for inclusion in a treatment plan comprises:

comparing the estimated CROPSS value against the lower threshold CROPSS value and if the estimated CROPSS value is less than the lower threshold CROPSS value then calculating a quantity of one or more of the water soluble cations from the first group and/or a quantity of one or more of the exchangeable cations from the first group for inclusion in a treatment plan, wherein the quantities are selected such that when added they generate an adjusted CROPSS value greater than or equal to the lower threshold CROPSS value.

In one form, the method further comprises:

comparing each of the measured exchangeable cation concentrations against a respective predetermined exchangeable cation threshold, and

for each cation in the first group, calculating a quantity of the respective exchangeable cation required to increase the exchangeable cation concentration to an adjusted cation concentration within the respective predetermined exchangeable cation threshold concentration for inclusion in a treatment plan,

wherein comparing each of the exchangeable cations against a respective predetermined exchangeable cation threshold may be performed before or after the step of estimating a CROPSS value, and if the step is performed before the step of estimating a CROPSS value, then the step of estimating a CROPSS value uses the adjusted exchangeable cation concentrations, and if the step is performed after the step of estimating a CROPSS, then the step of estimating a CROPSS is repeated using the adjusted exchangeable cation concentrations; and

the step of calculating a quantity of one or more of the water soluble cations from the first group and/or a quantity of one or more of the exchangeable cations from the first group comprises calculating at least a quantity of one or more of the water soluble cations from the first group.

In one form, y=Zeta Potential, x=CROPSS, and

the first group of cations consists of Calcium Magnesium, Potassium and Sodium and to two significant digits the predetermined mapping relationship is y=0.45x−42; or

the first group of cations consists of Calcium Magnesium, and Potassium and to two significant digits the predetermined mapping relationship is y=0.34x−42; or

the first group of cations consists of Calcium Magnesium, and Sodium and to two significant digits the predetermined mapping relationship is y=0.31x−41; or

the first group of cations consists of Calcium, Potassium and Sodium and to two significant digits the predetermined mapping relationship is y=0.30x−42; or

the first group of cations consists of Calcium and Magnesium and to two significant digits the predetermined mapping relationship is y=0.29x−51; or

the first group of cations consists of Calcium and Sodium and to two significant digits the predetermined mapping relationship is y=0.20x−43; or

the first group of cations consists of Calcium and Potassium and to two significant digits the predetermined mapping relationship is y=0.17x−41.

In one form, the method further comprises providing a treatment plan comprising the calculated at least a quantity of one or more of the water soluble cations from the first group and/or a quantity of one or more of the exchangeable cations from the first group.

According to a second aspect, there is provided a computer program product comprising instructions for causing one or more processors in a computer to perform a method for performing a soil analysis to determine quantities of one or more cations to be included in a soil treatment plan for an agricultural soil according to the first aspect.

According to a third aspect, there is provided a system for performing a soil analysis to determine quantities of one or more cations to be included in a soil treatment plan for an agricultural soil, the system comprising:

a soluble cation measurement apparatus for measuring a water soluble cation concentration in one or more received soil samples for a first group of cations comprising Calcium and at least one of Magnesium, Potassium and Sodium;

an exchangeable cation measurement apparatus for measuring an exchangeable cation concentration in the one or more received soil samples for the first group of cations;

a computing apparatus comprising a memory and at least one processor, the memory comprising instructions for causing the processor to perform a soil analysis method to determine quantities of one or more cations to be included in a soil treatment plan for an agricultural soil according to the first aspect.

According to a third aspect, there is provided a method for performing a soil analysis testing a soil sample to determine quantities of one or more cations to be included in a soil treatment plan for an agricultural soil, the method comprising:

obtaining one or more soil samples;

measuring a Zeta Potential of the one or more soil samples;

comparing the measured Zeta Potential with a predetermined threshold Zeta Potential, and if the measured Zeta Potential is less than the predetermined threshold Zeta Potential, then adding a quantity of one or more of the water soluble cations from a first group of cations comprising Calcium and at least one of Magnesium, Potassium and Sodium to the one or more soil samples;

re-measuring the Zeta Potential and comparing the re-measured Zeta Potential with the predetermined threshold Zeta Potential, and if the re-measured Zeta Potential is less than the predetermined threshold Zeta Potential, then adding a quantity of one or more of the water soluble cations from the first group of cations and/or a quantity of one or more of the exchangeable cations from the first group to the one or more soil samples, and repeating this step until the re-measured Zeta Potential is greater than or equal to the predetermined threshold Zeta Potential.

In one form, the method comprises preparing a treatment plan based upon the total quantity of the one or more of the water soluble cations from the first group of cations and the total quantity of the one or more of the exchangeable cations from the first group added to the one or more soil samples. In one form, the predetermined threshold Zeta Potential is in the range of −25 mV to −35 mV, and in a further form the predetermined threshold Zeta Potential is −30 mV.

### DESCRIPTION OF DRAWINGS

Embodiments of the present invention will be discussed with reference to the accompanying drawings wherein:

FIG. 1 is a flow chart of a method for performing a soil analysis to determine quantities of one or more cations to be included in a soil treatment plan for an agricultural soil according to an embodiment;

FIG. 2 is a flow chart of another method for performing a soil analysis to determine quantities of one or more cations to be included in a soil treatment plan based on the use of a CROPSS value according to an embodiment;

FIG. 3A is a plot of a first CROPSS value on the x axis vs Zeta Potential (mV) on the y axis for a range of soils according to an embodiment;

FIG. 3B is a plot of a second CROPSS value on the x axis vs Zeta Potential (mV) on they axis for a range of soils according to an embodiment;

FIG. 3C is a plot of a third CROPSS value on the x axis vs Zeta Potential (mV) on they axis for a range of soils according to an embodiment;

FIG. 3D is a plot of a fourth CROPSS value on the x axis vs Zeta Potential (mV) on the y axis for a range of soils according to an embodiment;

FIG. 3E is a plot of a fifth CROPSS value on the x axis vs Zeta Potential (mV) on the y axis for a range of soils according to an embodiment;

FIG. 3F is a plot of a sixth CROPSS value on the x axis vs Zeta Potential (mV) on they axis for a range of soils according to an embodiment;

FIG. 3G is a plot of a seventh CROPSS value on the x axis vs Zeta Potential (mV) on the y axis for a range of soils according to an embodiment;

FIG. 4 is schematic diagram of a computing apparatus according to an embodiment;

FIG. 5A is a first page of a soil report according to an embodiment;

FIG. 5B is a second page of a soil report according to an embodiment;

FIG. 5C is a third page of a soil report according to an embodiment;

FIG. 5D is a fourth page of a soil report according to an embodiment;

FIG. 5E is a fifth page of a soil report according to an embodiment;

FIG. 6A is a plot of exchangeable cation concentration vs Zeta Potential; and

FIG. 6B is a plot of soluble cation concentration vs Zeta Potential.

### DESCRIPTION OF EMBODIMENTS

Embodiments of an improved method for performing a soil analysis to enable development of a soil treatment plan (or management action) for an agricultural soil will now be described. The methods are based on an improved understanding of how the local soil structure can affect how plants uptake nutrients, and explain why previous treatment plans in which deficient nutrients are simply added are often ineffective. Specifically by making either direct measurements of a quantity known as the Zeta Potential or indirect estimates of Zeta potential using measurements of soluble and exchangeable cation concentrations, estimates of the quantities of one or more water soluble cations (Ca, Mg, K, Na) can be determined which result in adjustment of the Zeta Potential to a desirable range or value. As discussed herein, through investigations of the relationship between Zeta Potential and soluble and exchangeable cation concentrations (or ratios based upon these concentrations) a quantity referred to as a Cation Relationship of Plants and Soil Solution or CROPSS value has been developed along with a mapping of CROPSS to Zeta Potential to enable the CROPSS value to be used to determine the appropriate soil treatment to be applied that will adjust the Zeta Potential to a desired range or value.

To understand why previous attempts to develop soil treatment plans by bulk addition of nutrients, it is necessary to understand how plants uptake nutrients. Plants uptake nutrients from the soil by three major uptake pathways—namely Mass Flow, Diffusion and Root Interception. In each case plants require nutrients to be in the soil solution and utilise the soil solution as a medium to transport nutrients to the plant roots for mass flow and diffusion uptake and also to permit greater root exploration throughout the soil. The ions in soil solution are a regulated equilibrium of the ions attached to the cation and anion exchange sites in a soil system. That is all of the ions in soil solution interact and influence each other, and so impact on how the soil functions. However most soil testing fails to adequately measure or take into account these effects leading to ineffective treatment decisions based on measured values. Typically treatment decisions have largely been based on measured deficiencies of specific nutrients, for example based on comparing a measured exchangeable cation concentration with a threshold value, and then deciding what quantity of fertiliser to add to raise the deficient nutrient (or nutrients) to the required threshold level.

At the soil particle surface, the affinity for adsorbing nutrients is high due to the net negative charge of the soil particles. The further a nutrient is away from a soil particle the less affinity there is for the ions to be retained by the soil particles. Therefore there is a potential gradient that determines the difference between the affinity for a given ion to be retained by the soil or be released to the soil solution. This potential is the Zeta Potential of the soil (also known as the electrokinetic potential).

The Zeta Potential is the potential difference between the dispersion medium and the stationary layer of fluid attached to the dispersed particle and has previously been used in civil engineering applications to assess soil stability. Measuring the Zeta Potential (the electrical potential developed at the solid-liquid interface) of particles is a good indicator of their electrical potentials: The higher the Zeta Potential, the higher the surface potential of the charged clay particle. Higher Zeta Potential implies greater thickness of the electrical diffuse double layer of the particle. A value of the Zeta Potential of around 25-30 mV (positive or negative) can be taken as the arbitrary value that separates low-charge from high-charge surfaces. Its significance is that its value can be related to the stability of a colloidal dispersion (eg clay in soil solution), in that it indicates the degree of repulsion between adjacent, similarly charged particles in a dispersion. For particles that are small enough, a high Zeta Potential (eg—±40 mV or more) will confer soil stability: in other words, the dispersion will resist aggregation (which is undesirable in a soil). Conversely, when potential is low (i.e. from 0 to around ±30 mV), attraction exceeds repulsion and the dispersion will break down and flocculate or coagulate: the lower the potential, the faster the flocculation. Flocculation changes the physical characteristics of the suspension, and high flocculation, measurable as low Zeta Potential indicates improved structure of soils.

However agricultural soil testing laboratories do not typically measure Zeta Potential, nor do they measure water soluble cation concentrations which can be used to further understand nutrient accessibility. As outlined above, the Zeta Potential provides a measure of the general electrostatic environment or state of the soil which influences the ability of a plant to uptake the nutrients. For example whilst a certain nutrient may be nominally abundant according to traditional nutrient level guidelines, if the electrostatic environment is such that the nutrient is bound or unable to move, then the accessibility of that nutrient to the plant may actually be quite low. Previous treatment plans have typically focussed on adding bulk quantities of nutrients. Whilst such approaches may provide a long term store of nutrients, they do not necessarily affect or improve the immediate accessibility of the added nutrients to the soil solution which is accessed by plants. A suitable analogy is having a large bank account with a very low daily withdrawal limit—putting more money in the bank account provides long term security, but doesn't assist in paying off a large debt (or several debts) which is are currently due. Adding water soluble cations such as Calcium, Magnesium, Potassium and Sodium, can however improve the accessibility of cations, and change the electrostatic environment of the soil allowing plants to more easily access soil nutrients, including exchangeable cations provided by bulk nutrient addition. In the previous analogy adding water soluble cations immediately raises the withdrawal limit allowing all debts to be paid off. By choosing appropriate quantities of soluble and/or exchangeable cations based on knowledge of and/or their effect on the Zeta Potential (actual or estimated) more appropriate treatment plans can be developed and used compared to those based on nutrient abundance and/or exchangeable cation quantities/concentrations.

Thus one approach to improving soil treatment plans is to measure the Zeta Potential of a soil sample and use this measurement to guide selection of the quantities water soluble cations (Ca, Mg, K, Na) to be included in a treatment plan (which can then be implemented). Water soluble cations may be used as a stand alone treatment used to rapidly increase availability or accessibility of existing nutrients in the soil, or in conjunction with other nutrients/treatments such as quantities of bulk fertilisers containing specific nutrients (including exchangeable cations) as part of an overall treatment plan. For example the Zeta Potential can be compared with a threshold value, such as one in the range of −25 mV to −35 mV (eg −30 mV) which is indicative of the change from low charge to high charged surfaces. The threshold could also be specified as an absolute value such as |30|mV so that the acceptable (desired) range would be all values between the positive and negative boundary values—(−30 mV, 30 mV). In most agricultural soils the Zeta Potential will be less than zero so the threshold value can assumed to be a negative value and an acceptable value is thus greater than the threshold value (implicitly bounded by zero).

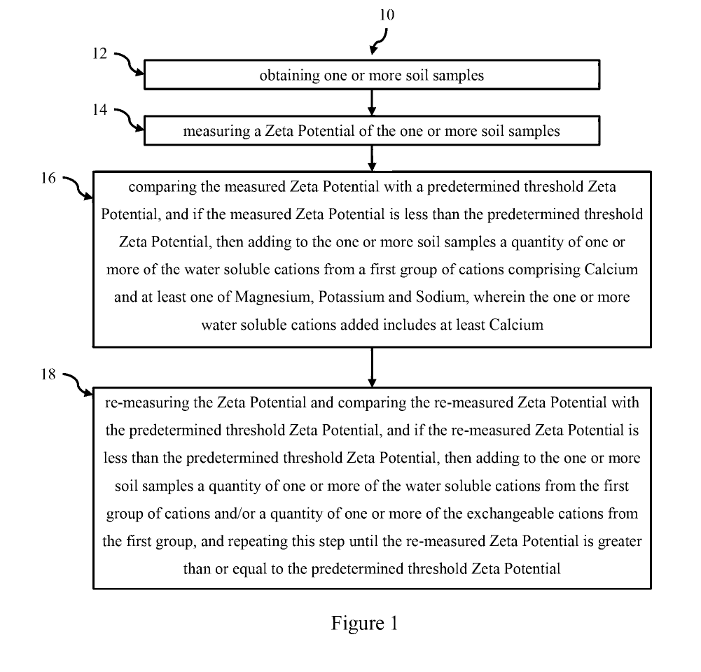

Referring now to FIG. 1, there is shown a flow chart of a method 10 for performing a soil analysis to determine quantities of one or more cations to be included in a soil treatment plan for an agricultural soil. The method begins at step 12 with obtaining one or more soil samples, and then measuring the Zeta Potential of the one or more soil samples 14. The Zeta Potential of a sample can be measured using Laser Doppler velocimetry, for example on a Malvern Zeta master Particle Electrophoresis Analyser.

Where there are more than one soil sample from a site of interest (eg a farm or field), separate Zeta Potential measurements may be performed on each soil sample. Multiple samples from the same site may be mixed and combined, and the Zeta Potential measurement performed on the combined samples. In embodiments where separate Zeta Potential measurements are taken of each soil sample, this may be used to generate sample location specific treatment plans to be generated and implemented, or alternatively, the separate individual measurements may be statistically averaged or combined, such as by using a statistical estimator such as a mean, weighted mean, median or other robust estimator. Further, an error estimate (eg standard deviation, interquartile range, etc) may also be obtained. Additionally, for a specific test sample (either single sample or multiple combined samples), repeated measurements of Zeta Potential could be performed by the measuring device and then statistically combined. Weighting factors can also be used to take into account sampling factors such as depth or area the sample corresponds to. For example multiple samples may be taken from a site, with one sample taken per paddock or plot, and weighting could compensate for differences in sizes of paddocks or plots across the site. In the context of the specification, measured Zeta Potential is intended to refer to both specific single measurements, as well as to a value obtained by statistically averaged or combined measurements from multiple samples (or measurements).

Once a measured Zeta Potential is obtained (which may be an average value), the measured Zeta Potential is compared with a pre-determined threshold Zeta Potential (step 16). In one embodiment, the threshold Zeta Potential is in the range of −25 mV to −35 mV, such as a value of −30 mV. Values in this range are indicative of the change from low charge to high charged surfaces, and as discussed above, in soils with Zeta Potentials of less than around −30 mV attraction exceeds repulsion and the dispersion will break down allowing flocculation or coagulation, improving the structure of soils and availability of nutrients and cations to plants (thus leading to increased yields). Threshold values outside of this range could be used, and the threshold value may represent a desired target value for the soil (−20, −15, −10), or a value desirable for an additional reason, for example to maximise the effect of another treatment applied as part of the soil treatment plan.

In this embodiment, an empirical approach is taken to determine the soil treatment. Thus if the measured Zeta Potential is less than the predetermined threshold Zeta Potential, then a quantity of one or more of the water soluble cations from a first group of cations comprising Calcium and at least one of Magnesium, Potassium and Sodium is added to the one or more soil samples. At step 18, the Zeta Potential is re-measured and the re-measured Zeta Potential is compared with the predetermined threshold Zeta Potential. If the re-measured Zeta Potential is less than the predetermined threshold Zeta Potential, then an additional quantity of the one or more of the water soluble cations from the first group of cations and/or a quantity of one or more of the exchangeable cations from the first group to the one or more soil samples is added. This step of adding, remeasuring and comparing is repeated until the re-measured Zeta Potential is greater than or equal to the predetermined threshold Zeta Potential. A treatment plan can then be prepared based upon the total quantity of the one or more of the water soluble cations from the first group of cations and the total quantity of the one or more of the exchangeable cations from the first group added to the one or more soil samples.

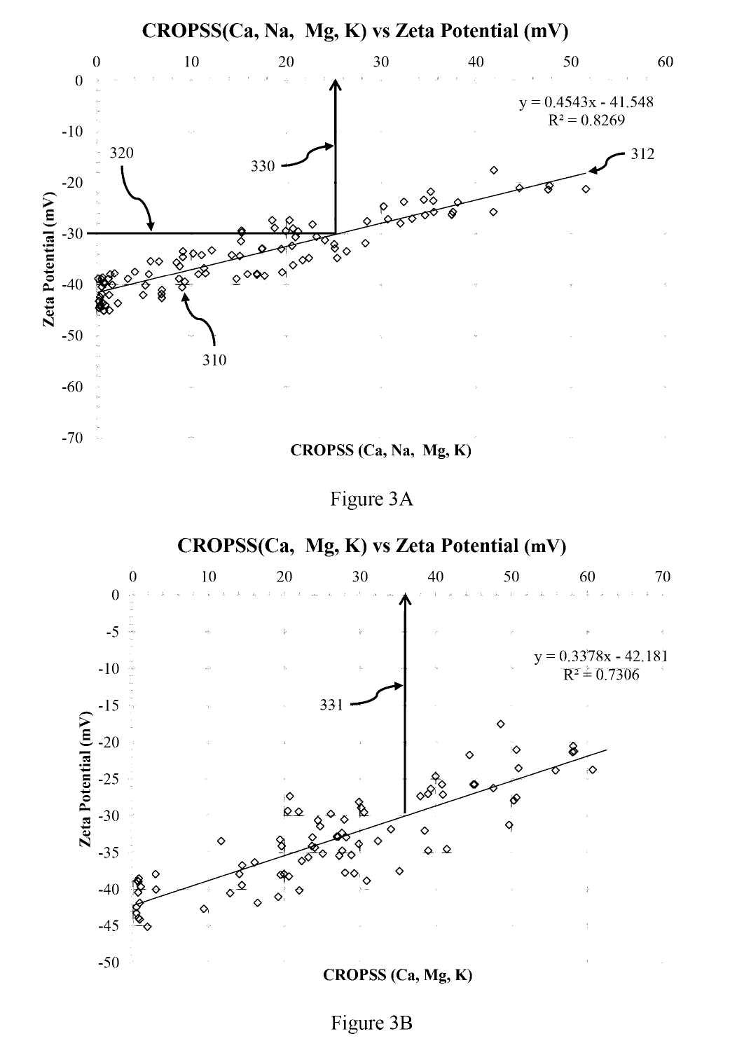

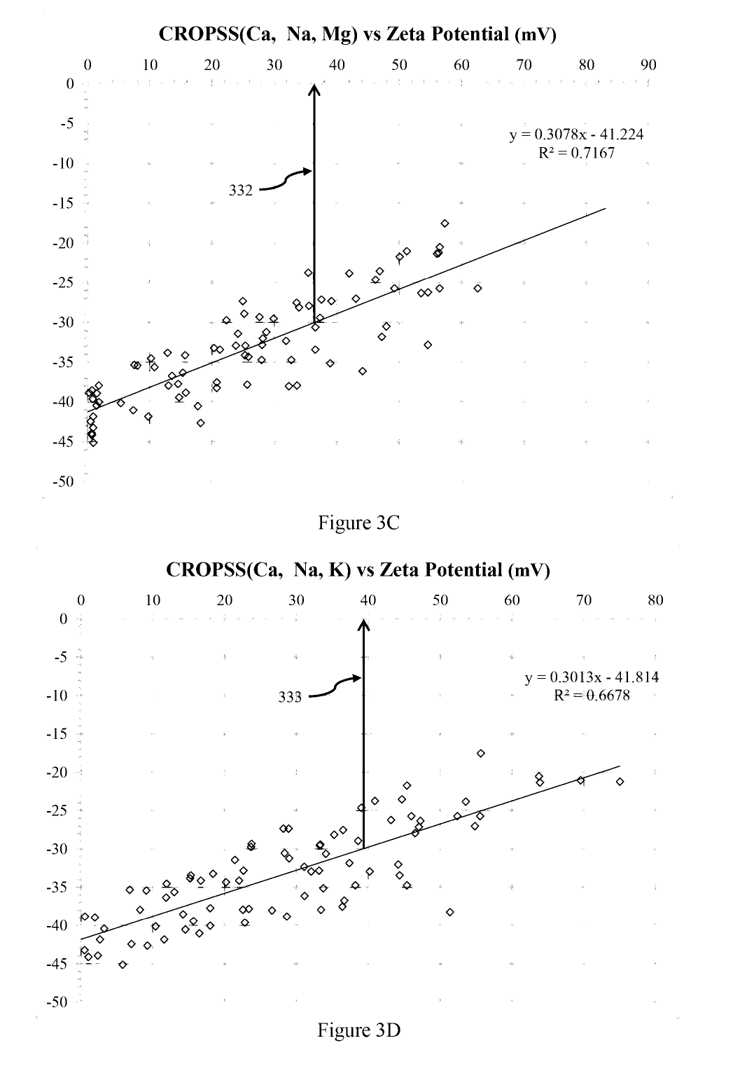

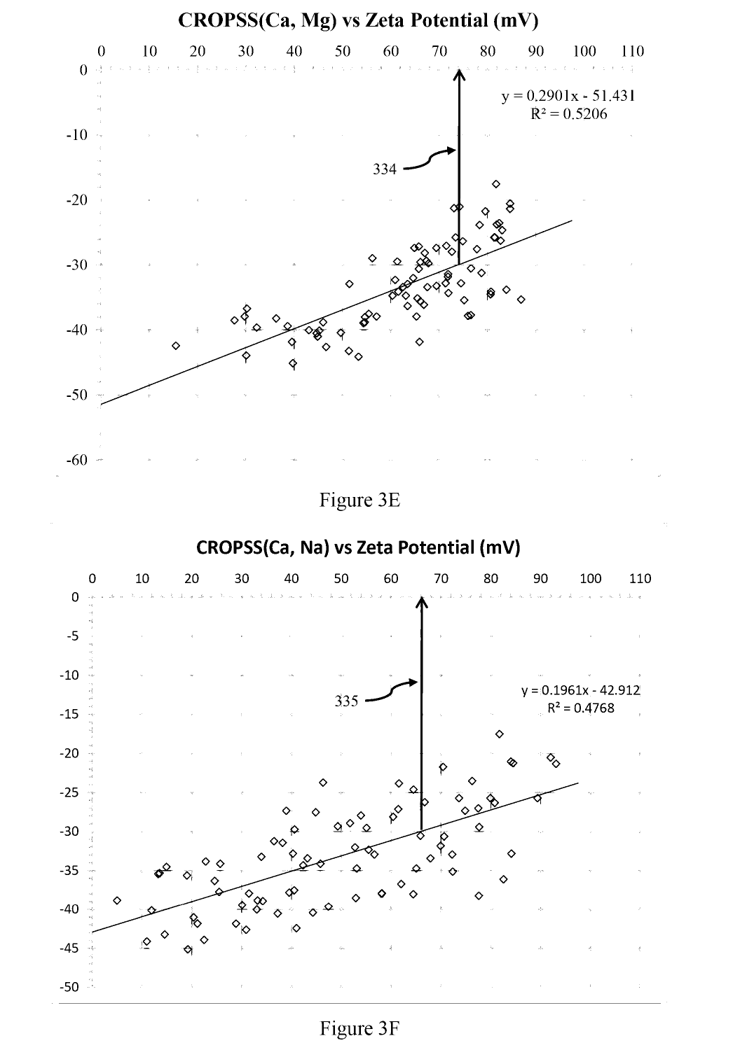

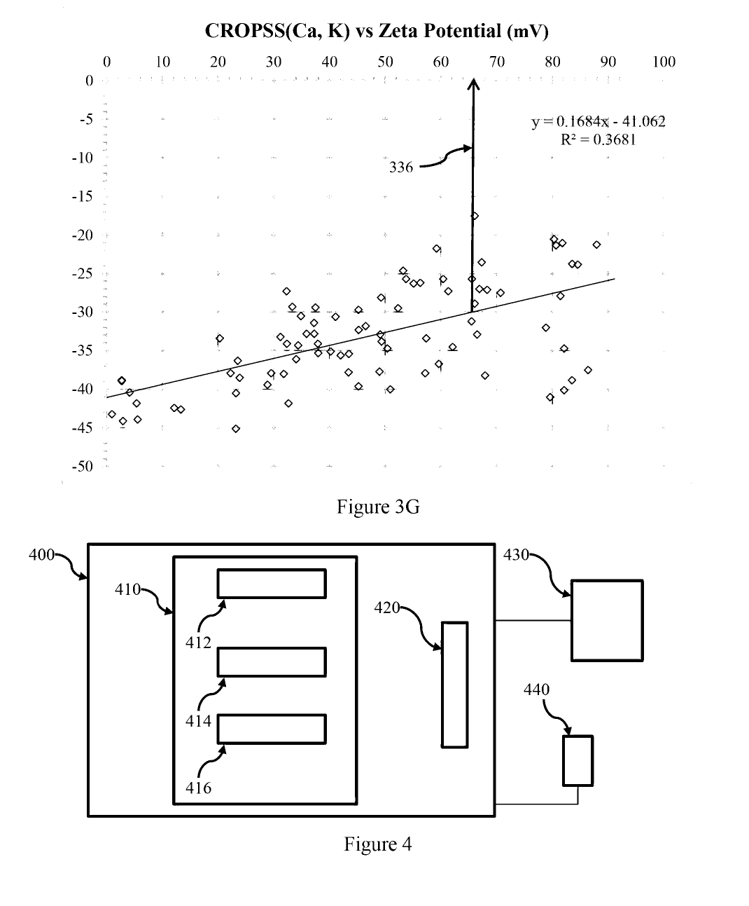

In this embodiment, the first addition includes at least water soluble Calcium ions, and may also include water soluble Magnesium, Potassium and Sodium cations. Water soluble cations are selected, as by being water soluble, they are able to rapidly change the Zeta Potential of the soil. This choice is further based on the results of correlations shown in FIGS. 3A to 3G which are discussed below, and Turbidity and related observations in relation to electrostatics of the group of cations comprising Calcium Magnesium, Potassium and Sodium. Turbidity is a measure of clay dispersion in solution, and it has been observed that the turbidity of soils in aqueous suspension decreases in the order Na>K>Mg>Ca. Further the negative charge of dispersed clays, measured as Zeta Potentials, decreases in the order Na>K>Mg>Ca, and it has been observed that divalent cations in solution induces larger clay particle size, and this affects the Zeta Potential of these clays. Hence the initial treatment comprises at least water soluble Calcium cations as this has the capability of rapidly changing the Zeta Potential. Other soluble cations (Mg, K, Na) and exchangeable cations can also be added at the same time. This may be based on additional information, such as measurements of other soil properties that may suggest additional treatments. If after the initial treatment, the Zeta Potential is still outside of the desired range (eg still less than, or more negative, than −30 mV) the requirement on the inclusion of Calcium can be relaxed and other combinations of water soluble cations, as well as exchangeable cations can be added. Further at each additional adding step, a different cation, or combination of cations can be selected. The quantities to add can be determined using a trial and error approach, or using a more guided or structured approach, such as one based on previous experiments or treatment plans. For example the first step may be a quantity of water soluble Calcium ions, and the second step may be quantity of water soluble Magnesium ions. In each case the Zeta Potential is remeasured to determine if the addition has been sufficient to lower the Zeta Potential to or below the target threshold.

As has been discussed above, soil testing laboratories do not measure or use Zeta Potential and most lack the required equipment to make such measurements. Whilst a trial and error experimental program to reduce the Zeta Potential as described above can be used to develop a treatment plan, an indirect method of estimating Zeta Potential is obtained based on measurements of soluble and exchangeable cation concentrations has been developed. As these embodiments do not require a direct measurement of Zeta Potential they can be implemented by soil testing laboratories that lack equipment to perform direct measurements of Zeta Potential. In this approach, a quantity referred to as the Cation Relationship of Plants and Soil Solution or CROPSS value was developed (or defined). When used with a mapping relationship between a CROPSS value and Zeta Potential for reference soils, a more guided approach to develop improved treatment plans.

The CROPSS value was developed to assist in understanding and linking the nutrients readily available in soil solution with the plant requirements for uptake via varying pathways, and to thereby provide a more guided approach to develop improved treatment plans. It uses typical measurements performed, or performable by soil analysis laboratories. The CROPSS value identifies the linkage between cations in solution driving the Zeta Potential of a soil and the distribution of the cations in solution that defines how high or low the Zeta Potential is. The understanding of this linkage provides insight into the availability primarily of cations such as Calcium, Magnesium, Potassium and Sodium, which influence the availability of counter-ions in solution and other plant essential nutrients. As is outlined below, there are several ways to define (and thus calculate) a CROPSS value, and each definition will produce a different mapping relationship between the specific definition of the CROPSS value and Zeta Potential. However irrespective of the specific definition of the CROPSS value, the CROPSS value and its mapping relationship to Zeta Potential provides a measure that can be used to understand the overall nutrient availability and uptake potential, and serve as a basis for developing treatment plans by allowing calculation of the quantities of one or more water soluble cations that result in adjustment of the Zeta Potential to a desirable range or value.

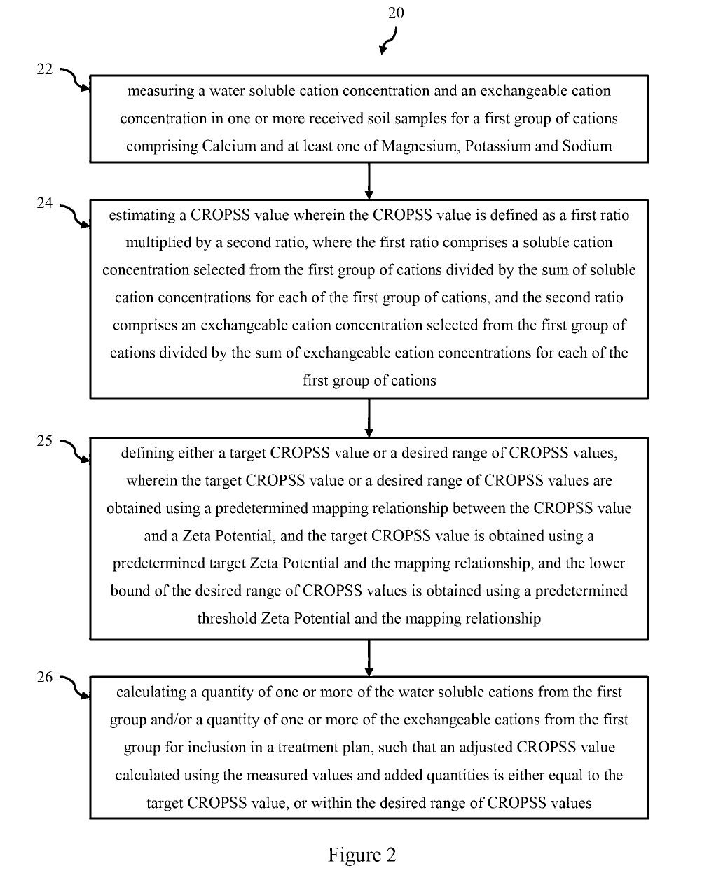

FIG. 2 is a flow chart of a method 20 for performing a soil analysis to determine quantities of one or more cations to be included in a soil treatment plan for an agricultural soil based on the use of a CROPSS value. The method comprises measuring a water soluble cation concentration and an exchangeable cation concentration in one or more received soil samples for a first group of cations comprising Calcium and at least one of Magnesium, Potassium and Sodium (step 22). A soluble cation measurement apparatus is used to measure the water soluble cation concentration for a first group of cations comprising Calcium and at least one of Magnesium, Potassium and Sodium. Suitable soluble cation measurement apparatus includes flame photometers, atomic absorption spectroscopic apparatus such as a flame-atomic absorption spectroscope, or an inductively coupled plasma-atomic emission spectroscope, or chromatographs such as an ion chromatograph. An exchangeable cation measurement apparatus is used to measure the exchangeable cation concentration for the first group of cations. Suitable exchangeable cation measurement apparatus include atomic absorption spectroscopic apparatus such as a flame-atomic absorption spectroscope, or an inductively coupled plasma-atomic emission spectroscope.

At step 24 a CROPSS value is estimated (or calculated). The CROPSS value is defined as a first ratio multiplied by a second ratio, where the first ratio comprises a soluble cation concentration selected from the first group of cations divided by the sum of soluble cation concentrations for each of the first group of cations, and the second ratio comprises an exchangeable cation concentration selected from the first group of cations divided by the sum of exchangeable cation concentrations for each of the first group of cations.

At step 25, a target CROPSS value or a desired range of CROPSS values is defined. In each case a mapping relationship between a CROPSS value and a Zeta Potential is used to define the range or the target CROPSS value. In the case of a target CROPSS value, this is obtained based on a predetermined target Zeta Potential (for example −20 mV) and the mapping relationship. In the case of a desired range, the lower bound of the desired range is obtained using a predetermined threshold Zeta Potential (eg −30 mV) and the mapping relationship. The upper boundary may be undefined (ie +∞), in which case the range is a one side range (ie only a lower bound is set), or the upper range may be based on the opposite sign predetermined Zeta Potential (eg +30 mV), or some other value such as zero Zeta Potential ie a desired range of Zeta Potentials from (−30, 0) or a smaller range of negative Zeta Potentials, eg (−25 mV, −10 mV). In each of these latter cases the mapping relationship is used to map the upper threshold Zeta Potential to an upper threshold CROPSS value for the desired range.

At step 26, a quantity of one or more of the water soluble cations from the first group and/or a quantity of one or more of the exchangeable cations from the first group is calculated for inclusion in a treatment plan. The quantities are selected such that when added they generate an adjusted CROPSS value within a predetermined threshold value, wherein the predetermined threshold value is based upon a predetermined Zeta Potential threshold and a mapping relationship between a CROPSS value and a Zeta Potential.

The CROPSS value is based on soluble cation concentrations and exchangeable cation concentrations for the group of cations containing Calcium and at least one of Magnesium, Potassium and Sodium. Water soluble cation and exchangeable cation concentrations are values which can be determined using apparatus available in most soil testing laboratories, and through the use of a mapping relationship between Zeta Potential and the CROPSS value, a suitable treatment plan can be determined without having to make Zeta Potential measurements on the soil samples. The CROPSS value can be generally defined as:

$$CROPSS = ( ( \frac{Water Soluble Cation {Concentration}_{i}}{{Σ}_{i} Water Soluble Cation {Concentration}_{i}} ) \times ( \frac{Exchangeable Cation {Concentration}_{i}}{{Σ}_{i} Exchangeable Cation {Concentration}_{i}} ) ) \\ where i ∈ \{ Ca and at least one of \{ Mg , K , Na \} \} \qquad Equation ( 1 )$$

A generic CROPSS value is defined as a first ratio multiplied by a second ratio and will thus vary from 0 to 1.0. If desired it can be further multiplied by 100 so that it can be expressed as a percentage value (ie in the range 0-100). In each ratio, a group of cations is defined as comprising Calcium and at least one of Magnesium, Potassium and Sodium. One of the group of cations is selected as the reference cation and is used as the numerator concentration. The denominator in each ratio is then the sum of group of Cations. For a specific definition of a CROPSS value a mapping relationship between a Zeta Potential and a CROPSS value (for that definition) can be determined. In one embodiment the mapping relationship is determined by performing a regression of CROPSS values against Zeta Potential measurements for a range of soils. FIGS. 3A to 3G show regression based mapping relationship of a range of specific CROPSS definitions. Units for the exchangeable and soluble cation concentrations are molar equivalent units C.mol/kg (which is also equivalent to meq/100 g). Concentrations can be converted into parts per million (ppm) or mg/L if desired. For example a concentration in C.mol/kg can be converted to ppm by multiplying by 200, 120, 390 and 230 for Ca, Mg, K and Na respectively.

If the calculated CROPSS value is less than a target CROPSS value or the lower bound of a desired range of CROPSS values, Equation (1) can be used to determine the effect on the CROPSS value of a treatment comprising adding one or more water soluble cations or exchangeable cations by inserting the new concentration values (post treatment) in to the equation and recalculating (by replacing Concentrationᵢ with (Concentrationᵢ+treatment)). Alternatively the amount of a cation treatment (either water soluble or exchangeable cation) to be added to increase the CROPSS value to a target CROPSS value can be calculated by rearranging the above equation. If xᵢ is the treatment amount for cation i, then this can be performed by setting (or replacing) the CROPSS value with the target CROPSS value, and replacing Concentrationᵢ with (Concentrationᵢ+x,), and then solving for xᵢ. This is relatively straightforward if the treatment is a single cation, and can be numerically solved or estimated where the treatment comprises more than one cation, such as by creating and solving a set of linear equations, or using Numerical techniques such as root estimation or Monte Carlo/trial and error. Such numerical estimation can be performed using a program such as MATLAB™ or a numerical library such as the NAG™ Numerical Library. For example if the treatment was to add an amount x of water soluble Calcium, then we could determine x by rearranging the following equation and solving for x:

$$TARGETCROPSS = ( ( \frac{Water Soluble Calcium {Concentration}_{i} + x}{x + {Σ}_{i} Water Soluble Cation {Concentration}_{i}} ) \times ( \frac{Exchangeable Calcium {Concentration}_{i}}{{Σ}_{i} Exchangeable Cation {Concentration}_{i}} ) ) \\ where i ∈ \{ Ca and at least one of \{ Mg , K , Na \} \} \qquad Equation ( 2 )$$

FIG. 3A is a plot of CROPSS on the x axis vs Zeta Potential (mV) on they axis for a range of soils (datapoints 310). In this embodiment the CROPSS value is defined using the group of cations comprising (Calcium, Magnesium, Potassium and Sodium) with Calcium as the reference cation:

$$CROPSS ( Ca , Mg , K , Na ) = ( ( \frac{Water Soluble Calcium Concentration}{\begin{matrix}{Σ}_{i ∈ \{ Ca , Mg , K , Na \}} Water Soluble \\ Cation {Concentration}_{i}\end{matrix}} ) \times ( \frac{Exchangeable Soluble Calcium Concentration}{\begin{matrix}{Σ}_{i ∈ \{ Ca , Mg , K , Na \}} Exchangeable Soluble \\ Cation {Concentration}_{i}\end{matrix}} ) ) \times 100 \qquad Equation ( 3 )$$

A linear regression was performed on individual data points 310 and the regression line 312 is plotted over the top. The regression equation is Zeta Potential=0.4543CROPSS −41.58 and has an R² value of 0.8269 indicating a strong positive correlation between Zeta Potential and CROPSS. This allows a threshold CROPSS value to be determined based upon a threshold Zeta Potential. As discussed above, a Zeta Potential of less than around −30 mV is desirable for agricultural soils. Entering a Zeta Potential of −30 mV in the regression equation generates a corresponding CROPSS ratio of 25.5. These values are indicated on the plot, with line 320 representing a Zeta Potential threshold of −30 mV intersects the regression line 312 at a CROPSS value of 25.5 which is indicated by vertical line 330. Thus if a soil sample returns a CROPSS value of less than 25.5 it is outside of the predetermined threshold Zeta Potential value and a treatment comprising selecting a quantity of one or more of the water soluble cations from the first group (Ca, Mg, K, Na) and/or a quantity of one or more of the exchangeable cations from the first group is selected so that after treatment the CROPSS value will be greater than or equal to 25.5 (the lower bound of the desired range).

Using the existing measurements and the target CROPSS value, it is then relatively straight forward algebra to determined additions which generate an adjusted CROPSS value within the desired range (ie ≥25.5). For example if the soluble concentration were 0.03, 0.02, 0.04 and 0.06 Cmol/kg for Ca, Mg, K and Na respectively, the first ratio in the CROPSS value is 0.2. If the exchangeable concentrations were 14, 1.75, 1.25 and 0.50 Cmol/kg for Ca, Mg, K and Na respectively then the second ratio is 0.8 and the CROPSS value is 16 (0.2×0.8×100). Increasing soluble Calcium alone will increase the first ratio and we can determine the amount of increase by dividing the target CROPSS ratio (25.5/100) by the second ratio (0.8) giving a target first ratio of 0.3185 (25.5/(0.8×100)). We can determine the amount to add (x) by solving (0.03+x)/(0.15+x)=0.3185 which gives a value of x of 0.0261 Cmol/kg. Thus adding 0.0261 Cmol/kg of water soluble Calcium will generate an adjusted CROPSS value greater of 25.5 (ie the target CROPSS value). Of course more Calcium could be added to achieve an even higher CROPSS value (and thus a less negative Zeta Potential). For example if the target CROPSS was 40, then this could be achieved by adding 0.09 Cmol/kg of water soluble Calcium.

In other embodiments both soluble and exchangeable cations could be added. In one embodiment the treatment plan could be developed based on first determining the amount of exchangeable cations to add and then the amount of the soluble cations to add. This could be performed by first comparing each of the measured exchangeable cation concentrations against a respective predetermined exchangeable cation threshold. These thresholds may correspond to existing thresholds used in current treatments. Then for each cation the quantity of the respective exchangeable cation required to increase the exchangeable cation concentration so that the adjusted cation concentration is within (eg greater than or equal to) the respective predetermined exchangeable cation threshold concentration can be calculated and included in the treatment plan. This addition of exchangeable cations will affect the CROPSS value. If a CROPSS value has not been estimated, then the adjusted exchangeable cations (rather than the measured exchangeable cations) concentrations are used in the estimation of the CROPSS value. Alternatively if the CROPSS value has already been calculated then it can be recalculated using adjusted exchangeable cation concentrations. This CROPSS value is compared with the desired CROPSS value, and then the quantity of one or more of water soluble cations to add is selected in order to generate a CROPSS value within the desired range (ie greater than or equal to the target CROPSS value).

Taking the above example, the recommended exchangeable cation concentrations for Mg and K may be 200 and 150 respectively. Increasing the amounts of the exchangeable cation concentrations for Mg and K to these values will alter the second ratio to 0.757, and the adjusted CROPSS ratio would change from 16 (0.2×0.8×100) to15.14 (0.2×0.757×100). Thus the amount of soluble Calcium to add must therefore increase according to (3+x)/(15+x)=(25.5/(0.757×100)=0.3369 giving an value of x of 3.1 mg/L of soluble Calcium.

As outlined above, a range of different CROPSS values can be defined based upon which specific cations are included in the first and second ratios. FIGS. 3B to 3G show a range of different CROPSS values, and the respective mapping relationship between Zeta Potential and the specific CROPSS value obtained from a linear regression. In FIGS. 3B to 3G, Calcium is always used as the reference cation (ie the numerator concentration) and the cations in the brackets represent the group of cations used in the CROPSS calculation. Plots 3B to 3D correspond to dropping one cation from the group—Na, K and Mg respectively, and Plots 3E to 3G correspond to groups comprising dropping two cations, or rather Calcium and one other cation—Mg, Na, and K respectively. Table 1 below lists the various CROPSS definitions and threshold values for a target Zeta of −30 mV for each of the CROPSS definitions plotted in FIGS. 3A to 3G. In each plot the regression line is illustrated and the respective CROPSS threshold listed in the last column of Table 1 is indicated (lines 331, 332, 333, 334, 335, 336 in FIGS. 3B, 3C, 3D, 3E, 3F, 3G respectively).

TABLE 1
Various CROPSS definitions and threshold values for a
target Zeta of −30 mV.

| | | | Threshold |
|---|---|---|---|
| Specific | Regression Equation | | CROPSS for |
| CROPSS definition | (y = Zeta, x = CROPSS) | R² | Zeta = −30 mV |
| $\frac{Ca}{\sum Ca + Mg + K + Na}$ | y = 0.4543x − 41.548 | 0.827 | 25.4 |
| $\frac{Ca}{\sum Ca + Mg + K}$ | y = 0.3378x − 42.181 | 0.731 | 36.1 |
| $\frac{Ca}{\sum Ca + Mg + Na}$ | y = 0.3078x − 41.224 | 0.717 | 36.5 |
| $\frac{Ca}{\sum Ca + K + Na}$ | y = 0.3013x − 41.814 | 0.668 | 39.2 |
| $\frac{Ca}{\sum Ca + Mg}$ | y = 0.2901x − 51.431 | 0.521 | 73.9 |
| $\frac{Ca}{\sum Ca + Na}$ | y = 0.1961x − 42.912 | 0.477 | 65.8 |
| $\frac{Ca}{\sum Ca + K}$ | y = 0.1684x − 41.062 | 0.368 | 65.7 |

As can be seen from Table 1, the strongest correlation occurs where the CROPSS value is defined using all four of the Cations in the group, and the correlation drops off as various cations are omitted (in the order Na, K, Mg). However even when Calcium is combined with only one other cation the correlation is still around 0.4-0.5. It will also be understood that other mapping relationships could be determined such as polynomial fitting, spline fitting, piecewise linear or non linear fitting methods, or other non-linear fitting methods. The mapping relationship could also be defined as a lookup table. It is also to be understood that a CROPSS value can be defined using a reference cation other than Calcium. Further whilst the CROPSS value is defined as the product of a soluble cation ratio and an exchangeable cation ratio (ie the product of two ratios), other variants and forms could be developed that utilise the measured soluble cation and measured exchangeable cation concentrations which is then mapped or correlated with Zeta Potential to generate similar mapping relationships. That is a mapping relationship between a function of measured soluble cation and measured exchangeable cation and the Zeta Potential.

Experimental trials were conducted on three crop types (Wheat, Barley, Canola) in NSW on the same soil type. The top 0-10 cm soil layer was analysed including measurements of the soluble Calcium concentration and Zeta Potential. The CROPSS value was calculated from the soil analysis data, and a management intervention (ie a CROPSS based treatment) was made to address and improve the low CROPSS value (ie 19.7 which is less than the desired threshold of 25) and large negative Zeta Potential (−43.8). Both the control site and the treatment site had the same base treatment of 80 Kg of Mono-Ammonium Phosphate (MAP) (Control). Additionally at the treatment site, a calcium dominated electrolyte solution (0.0230 cmol/0.1 m³) that was delivered into the soil at the same time as seed planting at a depth of around 8-12 cm. This treatment raised the soluble calcium concentration to 0.039 (0.016+0.023) resulting in a post treatment CROPSS value of 33.1 Table 2 lists the Zeta Potential and both pre and post CROPSS value (based on using each of Ca, Mg, K and Na) for the treatment site. Table 3 presents the yield results of the CROPSS based treatment of this field site listed in Table 2.

TABLE 2
Zeta Potential Measurement and CROPSS treatment at a field site in
NSW CROPSS Treatment - Calcium applied in
formulation = 0.0230 cmol/0.1 m³

| | Calcium | | | Calcium | |
|---|---|---|---|---|---|
| Depth | Soluble | CROPSS | Zeta | Soluble | CROPSS |
| (cm) | cmol Initial | initial | Potential | cmol Final | Final |
| 0-10 | 0.016 | 19.7 | −43.8 | 0.039 | 33.1 |

TABLE 3
Yield Results of CROPSS Based Treatment listed
in Table 2 at the NSW field site
Trial Results - CROPSS based treatment

| | Wheat | Barley | Canola |
|---|---|---|---|
| | t/ha | t/ha | t/ha |
| Control (80 Kg MAP) | 1.65 | 1.83 | 0.82 |
| CROPSS Treatment (plus 80 Kg MAP) | 1.82 | 2.07 | 0.94 |
| Yield Increase | 0.17 | 0.24 | 0.12 |
| LSD (P = .05) | 0.168 | 0.234 | 0.073 |
| Yield Increase Significant (at 0.05) | YES | YES | YES |

As can be seen in Table 3, the CROPSS based treatment was able to significantly increase the yield above the control (MAP) treatment. In other embodiments the CROPSS treatment could be performed at soil layer levels. In the above embodiment, the soil could have been portioned into a first 0-10 cm layer and a second 10-25 cm layer, and separate soil analysis performed on both layers. For example the second soil layer could have a Zeta Potential of −46.5 and an initial CROPSS of 19.50. The required treatment for each layer can then be calculated. A decision can then be made on whether to apply separate treatments in each layer, to separately raise the CROPSS value of each layer above a desired threshold, or a single treatment injected near the boundary of the layers could be used. The single treatment could be chosen to raise the CROPSS values for both layers over the threshold, or alternatively a treatment that raises at least one layer above the threshold and the other value close to the threshold (for example within 5%). For example a single treatment diluted across both layers may raise the CROPSS value of the top layer to 26 but only raise the CROPSS value of the bottom layer to 22. However with at least one layer above the threshold this may be sufficient to improve nutrient availability and increase yields.

The above treatment methods can be provided as a computer program product stored as instructions executable by a processor. The system may be a computer implemented system comprising of a display device, a processor and a memory and an input device. The memory may comprise instructions to cause the processor to execute a method described herein. The processor memory and display device may be included in a standard computing device, such as a desktop computer, a portable computing device such as a laptop computer or tablet, or they may be included in a customised device or system. The computing device may be a unitary computing or programmable device, or a distributed device comprising several components operatively (or functionally) connected via wired or wireless connections.

An embodiment of a computing device 400 is illustrated in FIG. 4 and comprises a central processing unit (CPU) 410, a memory 420, a display apparatus 430, and may include an input device 440 such as keyboard, mouse, etc. The CPU 410 comprises an Input/Output Interface 412, an Arithmetic and Logic Unit (ALU) 414 and a Control Unit and Program Counter element 116 which is in communication with input and output devices (eg input device 140 and display apparatus 130) through the Input/Output Interface. The Input/Output Interface may comprise a network interface and/or communications module for communicating with an equivalent communications module in another device using a predefined communications protocol (eg Bluetooth, Zigbee, IEEE 802.15, IEEE 802.11, TCP/IP, UDP, etc). A graphical processing unit (GPU) may also be included. The display apparatus may comprise a flat screen display (eg LCD, LED, plasma, touch screen, etc), a projector, CRT, etc. The computing device may comprise a single CPU (core) or multiple CPU's (multiple core), or multiple processors. The computing device may use a parallel processor, a vector processor, or be a distributed computing device including cloud based computing device(s). The memory is operatively coupled to the processor(s) and may comprise RAM and ROM components, and may be provided within or external to the device. The memory may be used to store the operating system and additional software modules or instructions. The processor(s) may be configured to load and executed the software modules or instructions stored in the memory to implement the method. In a cloud based computing system a user uses a local computing device to access a cloud based computer via a portal interface. In this case the calculations are performed remotely by the cloud based computing device(s) and provided to the user via their local computing device.

A computer program or computing system could be configured to obtain the measured water soluble and exchangeable cation concentrations—either by directly controlling measuring equipment or receiving data from measuring equipment such as over a communications link, receiving (and reading/parsing) a file containing the measurements, or even receiving the measurements via manual entry by a user (after having taken the appropriate measurements). Once the measurements are obtained the computer program instructions or system can perform the required estimation steps to generate a recommended treatment plan. In this case the computer program product could be provided with a mapping relationship between a CROPSS value and a Zeta Potential. The mapping relation could be provided as a regression relationship or a lookup table, or in some other similar form. The computer program or system may also produce a report containing a treatment plan, which can be provided to a user.

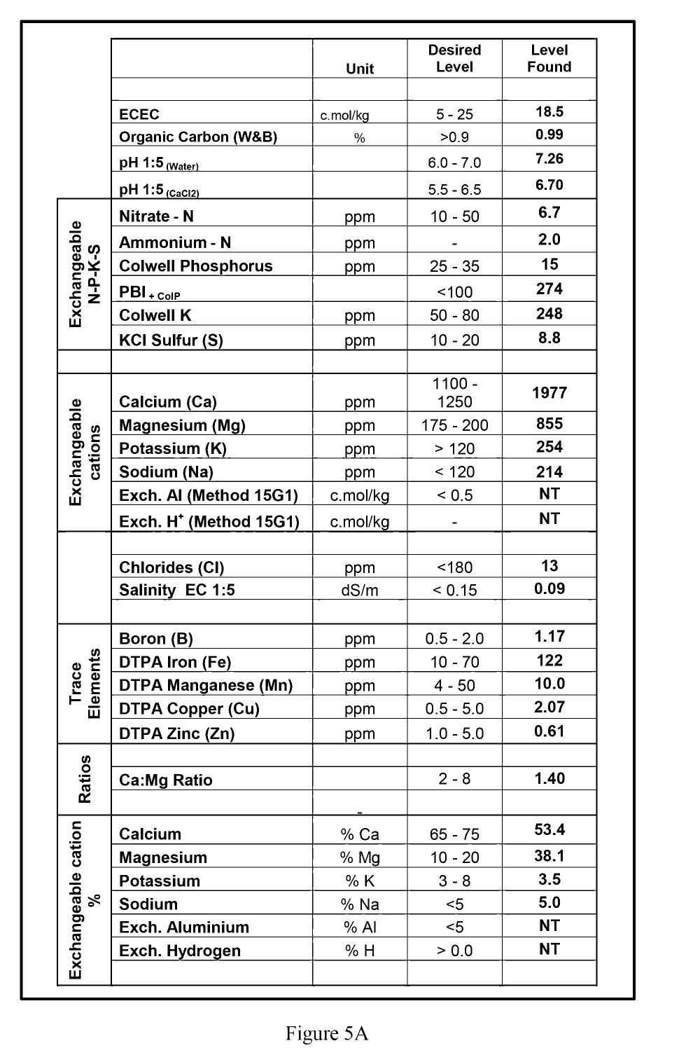

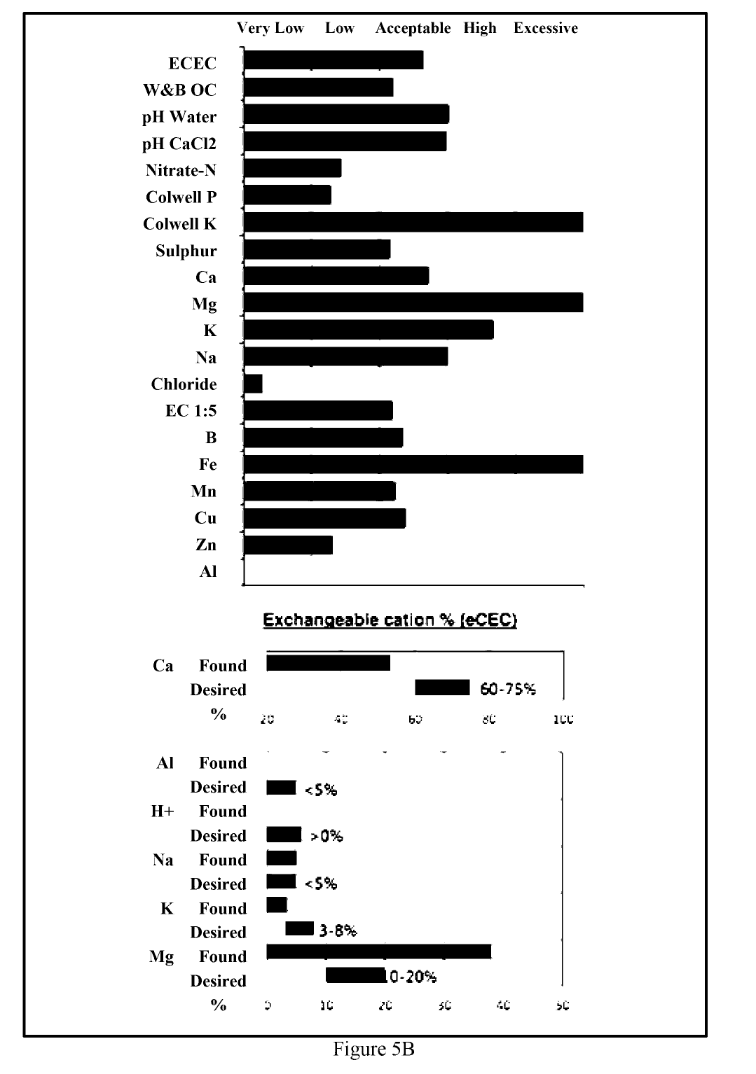

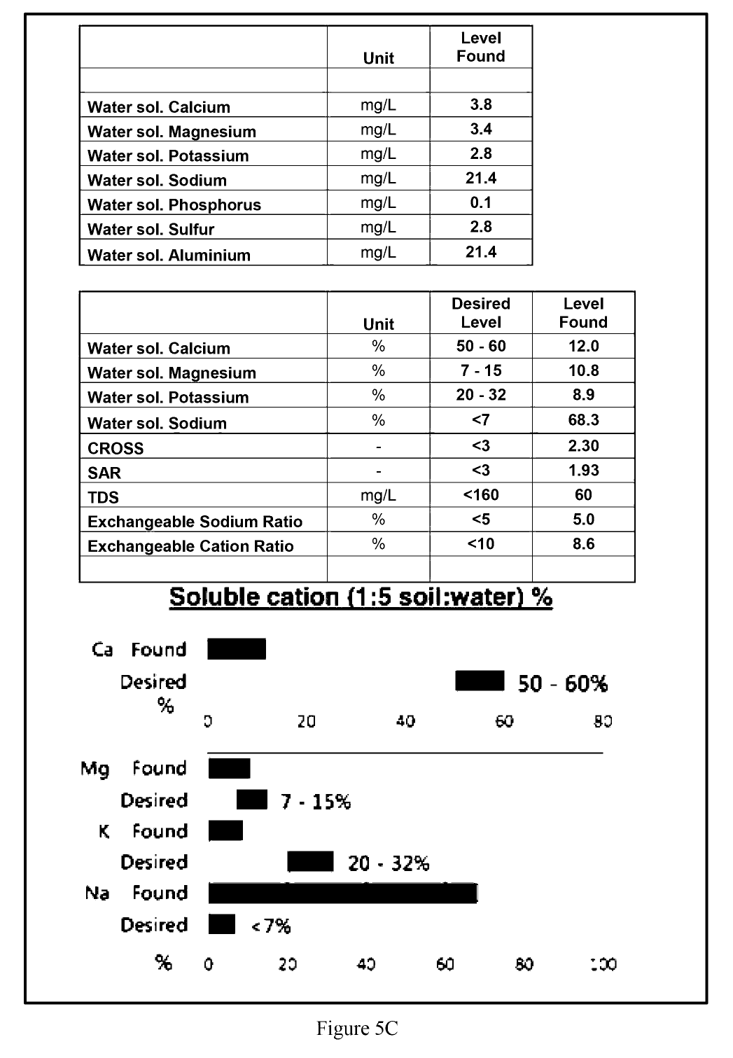

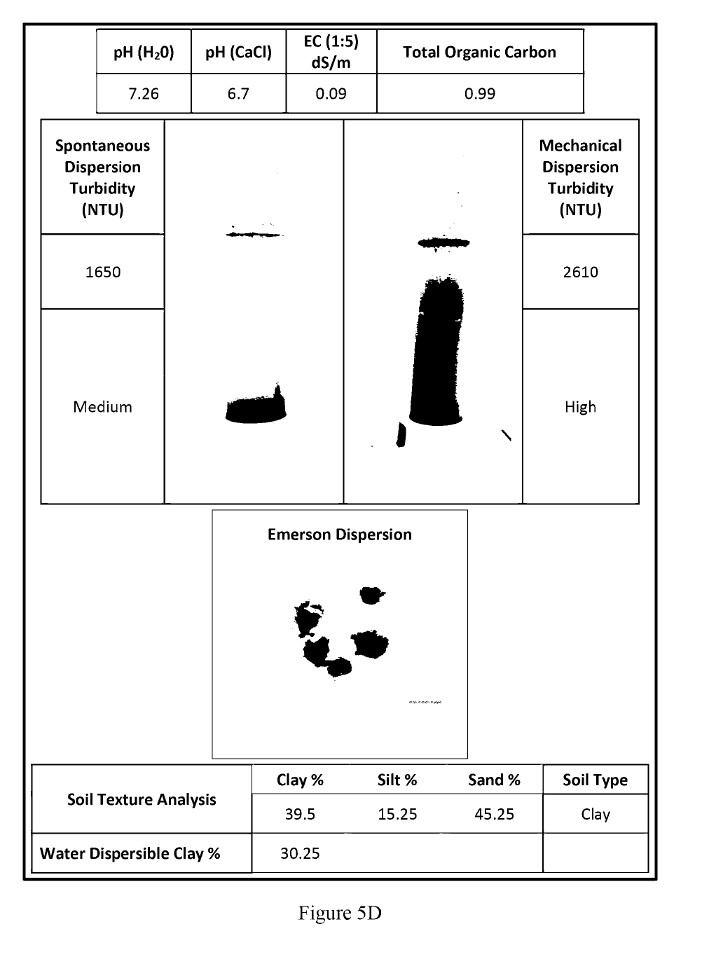

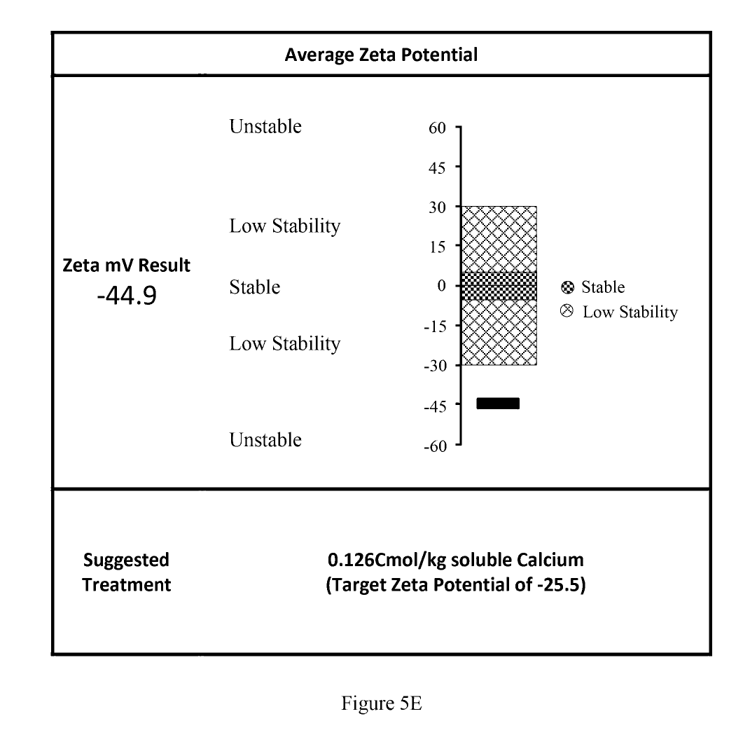

FIGS. 5A, B, C, D and E are the first, second, third, fourth and fifth sections of a soil report according to an embodiment. FIG. 5A list the exchangeable cation concentrations and FIG. 5B plots these with acceptability ranges (very low; low; acceptable; high; and excessive) along with the found and desired exchangeable cation percentages for Ca, Al, H+, Na, K and Mg. FIG. 5C list the water soluble cation concentrations and plots the found and desired soluble cation percentage for Ca, Mg, K and Na. FIG. 5D presents soil analysis measurements such turbidity and soil texture, and FIG. 5E plots the Zeta Potential measurements. From the information in the soil report, and linking the exchangeable and soluble cation concentrations via the CROPSS relationship, a recommendation can be made on the quantity of cations (eg Ca) to be applied into the water soluble pool of nutrients. The CROPSS value can be calculated using Calcium as reference and using all four cations (ie CROPSS(Ca, Mg, K, Na)) by first converting the exchangeable cations concentrations (FIG. 5A and 5B) and soluble cation concentrations (FIG. 5C) Calcium into cmol/kg units and inserting these into the equation in paragraph [0043] above. This yields a CROPSS=((0.019)/(0.019+0.028+0.007+0.093)) * ((9.865)/(9.865+7.125+0.65+0.93))*100=6.3. Putting this into the regression equation shown in FIG. 3A results in an estimate of the Zeta Potential of −41.5, which is close to the measured value of −44.9. A treatment based on increasing soluble calcium alone to raise the Zeta to −25.5 can then be calculated by rearranging the CROPSS equation: ((0.019+x)/(0.019+x+0.028+0.007+0.093)) * ((9.865)/(9.865+7.125+0.65+0.93))*100=25.5. Solving for x results in a treatment of 0.126 Cmol/kg of soluble calcium (see bottom of FIG. 5E).

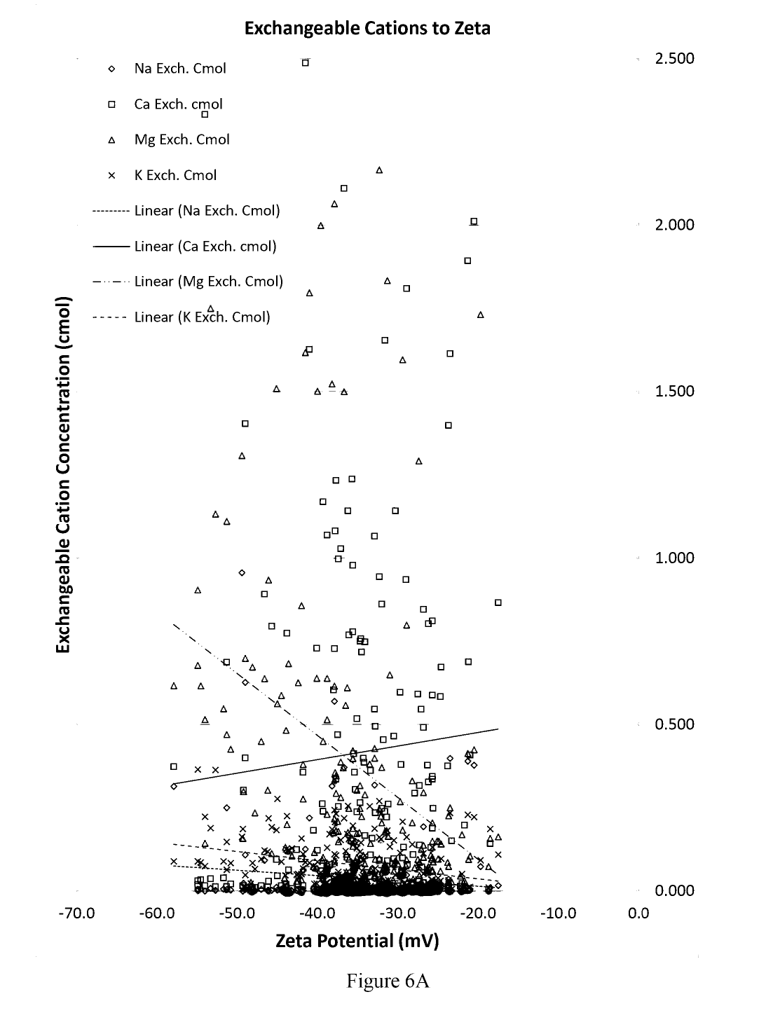

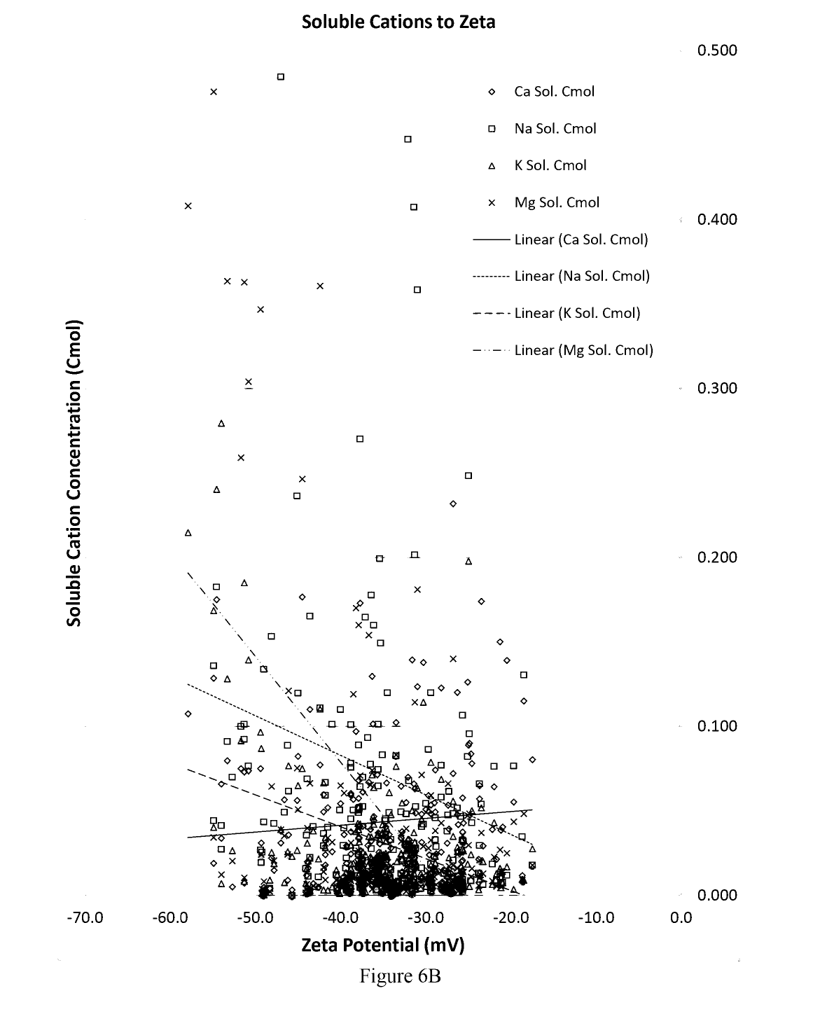

The CROPSS relationships described herein enable understanding of the linkage of how cations in solution drive the Zeta Potential of a soil. This linkage is not readily apparent from studying either the exchangeable cation concentration vs Zeta Potential, or soluble cation vs Zeta Potential. FIG. 6A is a plot of exchangeable cation concentration vs Zeta Potential for Ca, Na, Mg and K, and FIG. 6B is a plot of soluble cation concentration vs Zeta Potential for Ca, Na, Mg and K. In each case the concentration data for each cation shows high variability and poor correlation with Zeta potential (R² between 0 and 0.15). At best, the trend-lines indicate that the Calcium trend-line has a positive slope whereas the trend-lines for Sodium, Magnesium, and Potassium were negative, which may indicate that increasing calcium concentration may lower Zeta potential. In contrast the CROPSS relationship links combines both exchangeable cation concentrations with soluble cation concentrations, and links this to Zeta potential. As shown in FIGS. 3A to 3G, various CROPSS relationship can be used and show high correlation with Zeta. This enables the development of a treatment plan that does not require actual measurement of the Zeta Potential, thus allowing use in existing soil testing laboratories lacking apparatus to measure Zeta Potential.

As has been outlined above and shown herein, the distribution of cations in soil solution drives how high or low the Zeta Potential is, and this knowledge can be applied to develop improved soil treatment plans. In one method, the Zeta potential is measured and experimental adjusted by adding at least water soluble cations until the Zeta potential of the soil is within a desirable range, for example −30 to 0 (or even +30). Alternatively, by defining a CROPSS value as described herein based on water soluble cation and exchangeable cation concentrations, and determining a mapping relationship between a Zeta potential and the CROPSS value, a treatment plan can be developed that does not require actual measurement of the Zeta Potential, thus allowing use in existing soil testing laboratories lacking apparatus to measure Zeta Potential.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (eg, tangible media). In addition, for other aspects computer-readable media may comprise transitory computer-readable media (eg, a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (eg, RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

In one form the invention may comprise a computer program product for performing the method or operations presented herein. For example, such a computer program product may comprise a computer (or processor) readable medium having instructions stored (and/or encoded) thereon, the instructions being executable by one or more processors to perform the operations described herein. For certain aspects, the computer program product may include packaging material.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, the term “determining” encompasses a wide variety of actions. For example, “determining” may include calculating, computing, processing, deriving, investigating, looking up (eg, looking up in a table, a database or another data structure), ascertaining and the like. Also, “determining” may include receiving (eg, receiving information), accessing (eg, accessing data in a memory) and the like. Also, “determining” may include resolving, selecting, choosing, establishing and the like.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A method for performing a soil analysis to determine quantities of one or more cations to be included in a soil treatment plan for an agricultural soil, the method comprising:
measuring a water soluble cation concentration and an exchangeable cation concentration in one or more received soil samples for a first group of cations comprising Calcium and at least one of Magnesium, Potassium and Sodium;
estimating a Cation Relationship of Plants and Soil Solution (CROPSS) value wherein the CROPSS value is defined as a first ratio multiplied by a second ratio, where the first ratio comprises a soluble cation concentration selected from the first group of cations divided by the sum of soluble cation concentrations for each of the first group of cations, and the second ratio comprises an exchangeable cation concentration selected from the first group of cations divided by the sum of exchangeable cation concentrations for each of the first group of cations;
defining either a target CROPSS value or a desired range of CROPSS values, wherein the target CROPSS value or a desired range of CROPSS values are obtained using a predetermined mapping relationship between the CROPS S value and a Zeta Potential, and the target CROPSS value is obtained using a predetermined target Zeta Potential and the mapping relationship, and the lower bound of the desired range of CROPSS values is obtained using a predetermined threshold Zeta Potential and the mapping relationship;
calculating a quantity of one or more of the water soluble cations from the first group and/or a quantity of one or more of the exchangeable cations from the first group for inclusion in a treatment plan, such that an adjusted CROPSS value calculated using the measured values and added quantities is either equal to the target CROPSS value, or within the desired range of CROPSS values.

2. The method as claimed in claim 1, wherein the predetermined mapping relation is obtained by performing a regression of CROPSS values against Zeta Potential measurements for a range of soils.

3. The method as claimed in claim 1, wherein the predetermined Zeta Potential threshold is −30, and according to the mapping relationship the adjusted CROPSS value maps to a Zeta Potential greater than −30.

4. The method as claimed in claim 1, wherein the desired range is an open ended range such that the adjusted CROPSS value is within the desired range if the adjusted CROPSS value is greater than or equal to the lower threshold CROPSS value.

5. The method as claimed in claim 4, wherein the first group comprises Calcium and each of Magnesium, Potassium and Sodium, and the lower threshold CROPSS value is 25.5, and the first ratio comprises the soluble Calcium cation divided by the sum of soluble cation concentrations for each of the first group of cations, and the second ratio comprises the exchangeable Calcium cation concentration divided by the sum of exchangeable cation concentrations for each of the first group of cations.

6. The method as claimed in claim 1, wherein the step of calculating a quantity of one or more of the water soluble cations from the first group and/or a quantity of one or more of the exchangeable cations from the first group for inclusion in a treatment plan comprises:
comparing the estimated CROPSS value against the lower threshold CROPSS value and if the estimated CROPSS value is less than the lower threshold CROPSS value then calculating a quantity of one or more of the water soluble cations from the first group and/or a quantity of one or more of the exchangeable cations from the first group for inclusion in a treatment plan, wherein the quantities are selected such that when added they generate an adjusted CROPSS value greater than or equal to the lower threshold CROPSS value.

7. The method as claimed in claim 1, wherein the method further comprises:
comparing each of the measured exchangeable cation concentrations against a respective predetermined exchangeable cation threshold, and
for each cation in the first group, calculating a quantity of the respective exchangeable cation required to increase the exchangeable cation concentration to an adjusted cation concentration within the respective predetermined exchangeable cation threshold concentration for inclusion in a treatment plan, wherein comparing each of the exchangeable cations against the respective predetermined exchangeable cation threshold may be performed before or after the step of estimating a CROPSS value, and if the step is performed before the step of estimating a CROPSS value, then the step of estimating a CROPSS value uses the adjusted exchangeable cation concentrations, and if the step is performed after the step of estimating a CROPSS, then the step of estimating a CROPSS is repeated using the adjusted exchangeable cation concentrations,
and the step of calculating a quantity of one or more of the water soluble cations from the first group and/or a quantity of one or more of the exchangeable cations from the first group comprises calculating at least a quantity of one or more of the water soluble cations from the first group.

8. The method as claimed in claim 1, wherein y=Zeta Potential, x=CROPSS, and the first group of cations consists of Calcium Magnesium, Potassium and Sodium and to two significant digits the predetermined mapping relationship is y=0.45x−42; or
the first group of cations consists of Calcium Magnesium, and Potassium and to two significant digits the predetermined mapping relationship is y=0.34x−42; or
the first group of cations consists of Calcium Magnesium, and Sodium and to two significant digits the predetermined mapping relationship is y=0.31x−41; or
the first group of cations consists of Calcium, Potassium and Sodium and to two significant digits the predetermined mapping relationship is y=0.30x−42; or
the first group of cations consists of Calcium and Magnesium and to two significant digits the predetermined mapping relationship is y=0.29x−51; or
the first group of cations consists of Calcium and Sodium and to two significant digits the predetermined mapping relationship is y=0.20x−43; or
the first group of cations consists of Calcium and Potassium and to two significant digits the predetermined mapping relationship is y=0.17x−41.

9. The method as claimed in claim 1, further comprising providing a treatment plan comprising the calculated at least a quantity of one or more of the water soluble cations from the first group and/or a quantity of one or more of the exchangeable cations from the first group.

10. A non-transitory computer program product comprising instructions for causing one or more processors in a computer to perform a soil analysis method to determine quantities of one or more cations to be included in a soil treatment plan for an agricultural soil comprising:
receiving a measurement of a water soluble cation concentration and an exchangeable cation concentration in one or more received soil samples for a first group of cations comprising Calcium and at least one of Magnesium, Potassium and Sodium;
estimating a Cation Relationship of Plants and Soil Solution (CROPSS) value wherein the CROPSS value is defined as a first ratio multiplied by a second ratio, where the first ratio comprises a soluble cation concentration selected from the first group of cations divided by the sum of soluble cation concentrations for each of the first group of cations, and the second ratio comprises an exchangeable cation concentration selected from the first group of cations divided by the sum of exchangeable cation concentrations for each of the first group of cations;
defining either a target CROPSS value or a desired range of CROPSS values, wherein the target CROPSS value or a desired range of CROPSS values are obtained using a predetermined mapping relationship between the CROPSS value and a Zeta Potential, and the target CROPSS value is obtained using a predetermined target Zeta Potential and the mapping relationship, and the lower bound of the desired range of CROPSS values is obtained using a predetermined threshold Zeta Potential and the mapping relationship;
calculating a quantity of one or more of the water soluble cations from the first group and/or a quantity of one or more of the exchangeable cations from the first group for inclusion in a treatment plan, such that an adjusted CROPSS value calculated using the measured values and added quantities is either equal to the target CROPSS value, or within the desired range of CROPSS values.

11. A system for performing a soil analysis to determine quantities of one or more cations to be included in a soil treatment plan for an agricultural soil, the system comprising:
a soluble cation measurement apparatus for measuring a water soluble cation concentration in one or more received soil samples for a first group of cations comprising Calcium and at least one of Magnesium, Potassium and Sodium;
an exchangeable cation measurement apparatus for measuring an exchangeable cation concentration in the one or more received soil samples for the first group of cations;
a computing apparatus comprising a memory and at least one processor, the memory comprising instructions for causing the processor to perform a soil analysis method to determine quantities of one or more cations to be included in a soil treatment plan for an agricultural soil comprising:
receiving a measurement of a water soluble cation concentration from the soluble cation measurement apparatus and an exchangeable cation concentration from the exchangeable cation measurement apparatus in one or more received soil samples for a first group of cations comprising Calcium and at least one of Magnesium, Potassium and Sodium;
estimating a Cation Relationship of Plants and Soil Solution (CROPSS) value wherein the CROPSS value is defined as a first ratio multiplied by a second ratio, where the first ratio comprises a soluble cation concentration selected from the first group of cations divided by the sum of soluble cation concentrations for each of the first group of cations, and the second ratio comprises an exchangeable cation concentration selected from the first group of cations divided by the sum of exchangeable cation concentrations for each of the first group of cations;
defining either a target CROPSS value or a desired range of CROPSS values, wherein the target CROPSS value or a desired range of CROPSS values are obtained using a predetermined mapping relationship between the CROPSS value and a Zeta Potential, and the target CROPSS value is obtained using a predetermined target Zeta Potential and the mapping relationship, and the lower bound of the desired range of CROPSS values is obtained using a predetermined threshold Zeta Potential and the mapping relationship;
calculating a quantity of one or more of the water soluble cations from the first group and/or a quantity of one or more of the exchangeable cations from the first group for inclusion in a treatment plan, such that an adjusted CROPSS value calculated using the measured values and added quantities is either equal to the target CROPSS value, or within the desired range of CROPSS values.

12. A method for performing a soil analysis to determine quantities of one or more cations to be included in a soil treatment plan for an agricultural soil, the method comprising:
obtaining one or more soil samples;
measuring a Zeta Potential of the one or more soil samples;
comparing the measured Zeta Potential with a predetermined threshold Zeta Potential, and if the measured Zeta Potential is less than the predetermined threshold Zeta Potential, then adding to the one or more soil samples a quantity of one or more of the water soluble cations from a first group of cations comprising Calcium and at least one of Magnesium, Potassium and Sodium wherein the one or more water soluble cations added includes at least Calcium;
re-measuring the Zeta Potential and comparing the re-measured Zeta Potential with the predetermined threshold Zeta Potential, and if the re-measured Zeta Potential is less than the predetermined threshold Zeta Potential, then adding to the one or more soil samples a quantity of one or more of the water soluble cations from the first group of cations and/or a quantity of one or more of the exchangeable cations from the first group, and repeating this step until the re-measured Zeta Potential is greater than or equal to the predetermined threshold Zeta Potential.

13. The method as claimed in claim 12, further comprising:
preparing a treatment plan based upon the total quantity of the one or more of the water soluble cations from the first group of cations and the total quantity of the one or more of the exchangeable cations from the first group added to the one or more soil samples.

14. The method as claimed in claim 12, wherein the predetermined threshold Zeta Potential is in the range of −25 mV to −35 mV.

15. The method as claimed in claim 14, wherein the predetermined threshold Zeta Potential is −30 mV.

16. The non-transitory computer program product as claimed in claim 10, wherein the predetermined mapping relation is obtained by performing a regression of CROPSS values against Zeta Potential measurements for a range of soils.

17. The non-transitory computer program product as claimed in claim 10, wherein the predetermined Zeta Potential threshold is −30, and according to the mapping relationship the adjusted CROPSS value maps to a Zeta Potential greater than −30.

18. The non-transitory computer program product as claimed in claim 10, wherein the desired range is an open ended range such that the adjusted CROPSS value is within the desired range if the adjusted CROPSS value is greater than or equal to the lower threshold CROPS S value.

19. The non-transitory computer program product as claimed in claim 18, wherein the first group comprises Calcium and each of Magnesium, Potassium and Sodium, and the lower threshold CROPSS value is 25.5, and the first ratio comprises the soluble Calcium cation divided by the sum of soluble cation concentrations for each of the first group of cations, and the second ratio comprises the exchangeable Calcium cation concentration divided by the sum of exchangeable cation concentrations for each of the first group of cations.

20. The non-transitory computer program product as claimed in claim 10, wherein the step of calculating a quantity of one or more of the water soluble cations from the first group and/or a quantity of one or more of the exchangeable cations from the first group for inclusion in a treatment plan comprises:
comparing the estimated CROPSS value against the lower threshold CROPSS value and if the estimated CROPSS value is less than the lower threshold CROPSS value then calculating a quantity of one or more of the water soluble cations from the first group and/or a quantity of one or more of the exchangeable cations from the first group for inclusion in a treatment plan, wherein the quantities are selected such that when added they generate an adjusted CROPSS value greater than or equal to the lower threshold CROPSS value.

21. The non-transitory computer program product as claimed in claim 10, further comprising:
comparing each of the measured exchangeable cation concentrations against a respective predetermined exchangeable cation threshold, and
for each cation in the first group, calculating a quantity of the respective exchangeable cation required to increase the exchangeable cation concentration to an adjusted cation concentration within the respective predetermined exchangeable cation threshold concentration for inclusion in a treatment plan,
wherein comparing each of the exchangeable cations against the respective predetermined exchangeable cation threshold may be performed before or after the step of estimating a CROPSS value, and if the step is performed before the step of estimating a CROPSS value, then the step of estimating a CROPSS value uses the adjusted exchangeable cation concentrations, and if the step is performed after the step of estimating a CROPSS, then the step of estimating a CROPSS is repeated using the adjusted exchangeable cation concentrations,
and the step of calculating a quantity of one or more of the water soluble cations from the first group and/or a quantity of one or more of the exchangeable cations from the first group comprises calculating at least a quantity of one or more of the water soluble cations from the first group.

22. The non-transitory computer program product as claimed in claim 10 wherein y=Zeta Potential, x=CROPSS, and
the first group of cations consists of Calcium Magnesium, Potassium and Sodium and to two significant digits the predetermined mapping relationship is y=0.45x−42; or
the first group of cations consists of Calcium Magnesium, and Potassium and to two significant digits the predetermined mapping relationship is y=0.34x−42; or
the first group of cations consists of Calcium Magnesium, and Sodium and to two significant digits the predetermined mapping relationship is y=0.31x−41; or
the first group of cations consists of Calcium, Potassium and Sodium and to two significant digits the predetermined mapping relationship is y=0.30x−42; or
the first group of cations consists of Calcium and Magnesium and to two significant digits the predetermined mapping relationship is y=0.29x−51; or
the first group of cations consists of Calcium and Sodium and to two significant digits the predetermined mapping relationship is y=0.20x−43; or
the first group of cations consists of Calcium and Potassium and to two significant digits the predetermined mapping relationship is y=0.17x−41.

23. The non-transitory computer program product as claimed in claim 10, further comprising providing a treatment plan comprising the calculated at least a quantity of one or more of the water soluble cations from the first group and/or a quantity of one or more of the exchangeable cations from the first group.
